(19) United States
(12) Patent Application Publication
(10) Pub. No.: US 2015/0274626 A1
(43) Pub. Date: Oct. 1, 2015

(54) METHOD FOR PRODUCING METHACROLEIN AND METHACRYLIC ACID

(71) Applicant: MITSUBISHI RAYON CO.,LTD., Tokyo (JP)

(72) Inventors: Tetsufumi Yamaguchi, Otake-shi (JP); Tomomasa Tatsumi, Otake-shi (JP); Masahide Kondo, Otake-shi (JP); Yuji Fujimori, Otake-shi (JP)

(73) Assignee: MITSUBISHI RAYON CO.,LTD., Chiyoda-ku, Tokyo (JP)

(21) Appl. No.: 14/432,923

(22) PCT Filed: Oct. 29, 2013
(86) PCT No.: PCT/JP2013/079250

§ 371 Date: Apr. 1, 2015

(30) Foreign Application Priority Data
Nov. 7, 2012 (JP) 2012-245263

(51) Int. Cl. C07C 45/35; C07C 51/25

(52) U.S. Cl. CPC C07C 45/35; C07C 51/252

(57) ABSTRACT

Provided is a method for producing methacrolein and methacrylic acid which can improve yields of methacrolein and methacrylic acid. The method is to produce methacrolein and methacrylic acid through gas-phase contact oxidation of at least one raw material selected from the group consisting of isobutylene, TBA, and MTBE with molecular oxygen in the presence of a catalyst containing molybdenum, bismuth, and iron, using a fixed-bed reactor, in which, when a boundary temperature of activation energy of the oxidation reaction is denoted by TA (° C.), the reaction is started at a temperature lower than the reaction temperature TA (° C.), the reaction is controlled while increasing a reaction temperature such that a reaction rate of the raw material is kept constant, and the reaction is ended at a reaction temperature exceeding TA (° C.), and, when an average of rates of temperature increase until the reaction temperature reaches TA (° C.) is denoted by A (° C./hr) and an average of rates of temperature increase in the reaction temperature exceeding TA (° C.) is denoted by B (° C./hr), a ratio (A/B) of A and B is 0.05 to 0.18.

### TECHNICAL FIELD

The present invention relates to a method for producing methacrolein and methacrylic acid through gas-phase contact oxidation of at least one selected from the group consisting of isobutylene, tert-butyl alcohol (hereinafter, also described as TBA), and methyl-tert-butyl ether (hereinafter, also described as MTBE) using molecular oxygen in the presence of a catalyst.

### BACKGROUND ART

A method for producing methacrolein and methacrylic acid through gas-phase contact oxidation of isobutylene, tert-butyl alcohol, or methyl-tert-butyl ether on a catalyst containing molybdenum, bismuth, and iron as essential components has been widely known and also has been industrially used. The reaction is carried out, for example, using a fixed-bed multitubular reactor at the range of 300 to 400° C.

Such a catalyst which is used in the gas-phase contact oxidation reaction is used for a relatively long period of time. However, since activity of the catalyst is lowered with time, a reaction rate of a raw material is lowered with time. Therefore, in order to maintain the reaction rate of the raw material, a method of increasing a reaction temperature and then performing an operation has been employed.

Meanwhile, various methods have been suggested for the purpose of using a catalyst for a longer period of time. For example, Patent Document 1 discloses a method for regenerating a catalyst of which the activity has been lowered due to the use, under atmosphere substantially composed of air at 380 to 540° C. Patent Document 2 discloses a method in which, when a boundary temperature of activation energy of a reaction for producing methacrolein and methacrylic acid using a catalyst is denoted by T (° C.), the reaction is started at a temperature equal to or lower than T−3 (° C.), the reaction is continued while the reaction temperature is caused to be increased in accordance with lowering in catalyst activity, and activation treatment is performed at least once on the catalyst before the reaction temperature becomes higher than T. Patent Document 3 discloses a method in which, when a boundary temperature of activation energy of a reaction is denoted by TA (° C.), a reaction pressure or a molar ratio of oxygen to a raw material is changed to be controlled such that a reaction rate of the raw material is kept constant in a temperature range of from TA−15 (° C.) to TA (° C.).

### CITATION LIST

### Patent Document

Patent Document 1: JP 5-29502 B
Patent Document 2: JP 11-263739 A
Patent Document 3: WO 2007/105523 A

### SUMMARY OF THE INVENTION

### Problem to be Solved by the Invention

However, the methods described in these Patent Documents are effective for using the catalyst for a long period of time, but are not sufficient for the yields (selectivities) of methacrolein and methacrylic acid in the period of using the catalyst. Therefore, further improvement is required.

Further, from the industrial point of view, there is a demand to develop a method for producing methacrolein or the like which can maximize the yield (selectivity) in the entire period of using the catalyst without decreasing the period of using the catalyst in consideration of both of an operation in a period from a reaction start temperature until the temperature reaches a boundary temperature of activation energy and an operation in a period from the boundary temperature of activation energy until the temperature reaches a reaction end temperature exceeding the boundary temperature.

An object of the invention is to provide a method for producing methacrolein and methacrylic acid which can improve yields of methacrolein and methacrylic acid.

### Means for Solving Problem

The method for producing methacrolein and methacrylic acid according to the invention is a method for producing methacrolein and methacrylic acid through gas-phase contact oxidation of at least one raw material selected from the group consisting of isobutylene, tert-butyl alcohol, and methyl-tert-butyl ether with molecular oxygen in the presence of a catalyst containing molybdenum, bismuth, and iron, using a fixed-bed reactor, in which, when a boundary temperature of activation energy of the oxidation reaction is denoted by TA (° C.), the reaction is started at a temperature lower than the reaction temperature TA (° C.), the reaction is controlled while increasing a reaction temperature such that a reaction rate of the raw material is kept constant, and the reaction is ended at a reaction temperature exceeding TA (° C.), and, when an average of rates of temperature increase until the reaction temperature reaches TA (° C.) is denoted by A (° C./hr) and an average of rates of temperature increase in the reaction temperature exceeding TA (° C.) is denoted by B (° C./hr), a ratio (AB) of A and B is 0.05 to 0.18.

### Effect of the Invention

According to the method related to the invention, it is possible to improve yields of methacrolein and methacrylic acid.

### MODE(S) FOR CARRYING OUT THE INVENTION

### Catalyst and Method for Producing Same

A catalyst, which is used in the invention, is not particularly limited as long as it contains molybdenum, bismuth, and iron, but the catalyst preferably has the composition represented by the following Formula (1). Moreover, the catalyst according to the invention may be a composite oxide.

MoₐBi_{b}Fe_{c}M_{d}XₑY_{f}Z_{g}SiₕOᵢ (1)

In the above Formula (1), Mo represents molybdenum, Bi represents bismuth, Fe represents iron, Si represents silicon, and O represents oxygen. M represents at least one element selected from the group consisting of cobalt and nickel. X represents at least one element selected from the group consisting of chromium, lead, manganese, calcium, magnesium, niobium, silver, barium, tin, tantalum, and zinc. Y represents at least one element selected from the group consisting of phosphorus, boron, sulfur, selenium, tellurium, cerium, tungsten, antimony, and titanium. Z represents at least one element selected from the group consisting of lithium, sodium, potassium, rubidium, cesium, and thallium. Each of a, b, c, d, e, f, g, h, and i represents an atomic ratio of each element, and when a is 12, b is 0.01 to 3, c is 0.01 to 5, d is 1 to 12, e is 0 to 8, f is 0 to 5, g is 0.001 to 2, and h is 0 to 20. i is an atomic ratio of oxygen necessary for satisfying a valence of each of the components. Incidentally, the composition of the catalyst is a value calculated from an amount of the charged raw material of each element.

Hereinafter, a method for producing a catalyst, which is used in the invention, will be described.

The method for producing a catalyst according to the invention is not particularly limited, and various methods can be employed. For example, a method for obtaining a catalyst by drying an aqueous slurry containing at least molybdenum, bismuth, and iron, pulverizing and molding if necessary, and then subjecting heat treatment may be exemplified.

The method for producing an aqueous slurry containing at least molybdenum, bismuth, and iron is not particularly limited, and various methods such as a coprecipitation method and an oxide mixing method can be used.

Raw materials of catalyst components used for producing an aqueous slurry are not particularly limited, and examples thereof may include an oxide, chloride, hydroxide, sulfate, nitrate, carbonate, acetate, or ammonium salt of each element, or a mixture thereof. For example, as a raw material of molybdenum, ammonium paramolybdate, molybdenum trioxide, or the like can be used. As a raw material of bismuth, bismuth oxide, bismuth nitrate, or the like can be used. As a raw material of iron, ferric nitrate, ferric oxide, or the like can be used. One kind of these may be used, or two or more kinds thereof may be used in combination.

The method of drying the aqueous slurry is not particularly limited, and examples thereof include a drying method using a spray drying machine, a drying method using a slurry dryer, a drying method using a drum dryer, and a method of pulverizing a lump dry matter obtained by evaporation to dryness. As for drying conditions, for example, in a case where drying is performed by using a spray drying machine, the inlet temperature is preferably 100 to 500° C. and more preferably 110 to 300° C. Further, the outlet temperature is preferably 100 to 300° C. and more preferably 105 to 200° C.

There is a case where the dry powder obtained in this manner contains a salt of nitric acid or the like derived from the catalyst raw material. In this case, when calcination is performed after molding of the dry powder, the salt may be decomposed and thus the strength of the molded product may be decreased in some cases. For this reason, it is preferable to perform the calcination after drying. The primary calcination may be performed for the purpose of the desorption of the salt and the secondary calcination for forming a final catalyst active center structure may be performed after the molding which will be described later is performed. Moreover, after the primary calcination and the secondary calcination are performed, the molding may be carried out. The primary calcination and the secondary calcination may be performed as a single calcination by combining them or may be performed separately. The calcination conditions are not particularly limited, and well-known conditions can be employed. For example, the calcination can be performed at the temperature range of 200 to 600° C. for 0.5 to 10 hours. In a case where the primary calcination and the secondary calcination are performed, the temperature of the primary calcination is preferably 200 to 400° C., and more preferably 250 to 350° C. The time of the primary calcination is preferably 0.5 to 2 hours. The temperature of the secondary calcination is preferably 400 to 550° C., and more preferably 500° C. to 520° C. The time of the secondary calcination is preferably 2 to 5 hours.

The method for molding dry powder before calcination or after calcination is not particularly limited, and various molding methods such as tablet molding, extrusion molding, and supporting on a carrier can be used. Further, on the occasion of molding, for the purpose of controlling a specific surface area, pore volume, and pore distribution of a molded product, increasing mechanical strength, or enhancing handleability at the time of molding, an inorganic compound such as graphite or diatomaceous earth, inorganic fiber such as glass fiber, ceramic fiber, or carbon fiber, and an organic binder such as methylcellulose, ethylcellulose, carboxyl methylcellulose, hydroxy ethylcellulose, hydroxyethyl methylcellulose, hydroxypropyl cellulose, or hydroxypropyl methylcellulose may be added. One kind of these may be used, or two or more kinds thereof may be used in combination.

The shape of the molded product is not particularly limited, and examples thereof may include a spherical shape, a columnar shape, a ring shape, and a star shape. Further, in a case where the molding is performed by supporting on a carrier, it is possible to use, as a carrier, silica, alumina, silica-alumina, magnesia, titania, silicon carbide, or the like. One kind of these may be used, or two or more kinds thereof may be used in combination.

(Method for Producing Methacrolein and Methacrylic Acid)

The method for producing methacrolein and methacrylic acid according to the invention is a method for producing methacrolein and methacrylic acid through gas-phase contact oxidation of at least one raw material selected from the group consisting of isobutylene, tert-butyl alcohol, and methyl-tert-butyl ether with molecular oxygen in the presence of a catalyst containing molybdenum, bismuth, and iron, using a fixed-bed reactor, in which, when a boundary temperature of activation energy of the oxidation reaction is denoted by TA (° C.), the reaction is started at a temperature lower than the reaction temperature TA (° C.), the reaction is controlled while increasing a reaction temperature such that a reaction rate of the raw material is kept constant, and the reaction is ended at a reaction temperature exceeding TA (° C.), and, when an average of rates of temperature increase until the reaction temperature reaches TA is denoted by A (° C./hr) and an average of rates of temperature increase in the reaction temperature exceeding TA is denoted by B (° C./hr), a ratio (A/B) of A and B is 0.05 to 0.18.

It is known in a gas-phase contact reaction using a solid catalyst that the target activation energy of the reaction often shows different values in each of a low reaction temperature region and a high reaction temperature region with a certain reaction temperature being a boundary. For example, it is reported in JOURNAL OF CATALYSIS, Vol. 41, pp. 134 to 139 that such different activation energies as those described above are observed in the reaction of gas-phase contact oxidation of 1-butane on a catalyst composed of a composite oxide including molybdenum and bismuth. The reason why such a phenomenon is observed is because the rate-determining step of the reaction is different depending on the reaction temperature, which is described in detail in Shokubai Koza (Catalysis Course), Vol. 1, Chapter 4, Kodansha Ltd. (edited by Catalysis Society of Japan). It is presumed that a reaction of reactant molecules on the catalyst active center be the rate-determining step in the low reaction temperature region while diffusion of reactant molecules to the catalyst active center be the rate-determining step in the high reaction temperature region.

The present inventors have analyzed the activation energy of the reaction that produces methacrolein and methacrylic acid through gas-phase contact oxidation of at least one raw material selected from the group consisting of isobutylene, tert-butyl alcohol, and methyl-tert-butyl ether with molecular oxygen in the presence of a catalyst containing molybdenum, bismuth, and iron, using a fixed-bed reactor, and they have confirmed that the activation energy shows different values in each of a low reaction temperature region and a high reaction temperature region with a boundary temperature TA (° C.) of the activation energy being a boundary.

In the invention, the boundary temperature TA (° C.) of the activation energy is obtained as follows. First, a catalyst is packed in a reaction tube equipped with a thermo-bath, and a temperature at which a heat medium is introduced to the thermo-bath (reaction temperature) is changed in the range of 315 to 375° C. at an interval of 2 to 5° C., thereby obtaining a reaction rate of the raw material at each temperature. Here, the reaction rate is obtained by the following equation.

Reaction rate (%) of raw material=M/N×100

(M represents the number of moles of the raw material reacted and N represents the number of moles of the raw material supplied.)

Subsequently, a reaction rate constant is obtained by the following equation.

K=(SV)×(1/ρ)×ln[100/(100−X)]

(K represents a reaction rate constant, SV represents a space velocity, ρ represents a packing density of the catalyst, and X represents a reaction rate (%) of the raw material.)

Subsequently, 1/T is plotted as the horizontal axis and ln K is plotted as the vertical axis, and after each item of data is plotted, two approximation straight lines are drawn and inclinations of these lines are obtained. Here, 1/T represents a reciprocal of the temperature at which a heat medium is introduced to the thermo-bath of the reaction tube (reaction temperature, absolute temperature) and ln K represents a natural logarithm of the reaction rate constant. The approximation straight line can be obtained by the least-square method. A value obtained by multiplying the absolute value of the inclination of the obtained approximation straight line by a gas constant is the activation energy to be obtained, and the reciprocal of the value on the lateral coordinates of the intersection of the two approximation straight lines is the boundary temperature TA of the activation energy to be obtained.

The boundary temperature TA is obtained using isobutylene as a reaction raw material. This is because, even when TBA is used as a reaction raw material instead of isobutylene, TBA is decomposed rapidly in isobutylene and water on a catalyst containing molybdenum, bismuth, and iron and thus the reaction pattern thereof is substantially the same as the oxidation reaction of isobutylene. Therefore, when TBA is used as a reaction raw material, the boundary temperature TA of the activation energy of the reaction in the case of using isobutylene as a raw material can also be used without any change. In addition, when MTBE is used as a raw material, in similar to the case of using TBA, the same fact as in the oxidation reaction of isobutylene can also be considered and thus the boundary temperature TA of the activation energy of the reaction in the case of using isobutylene as a raw material can also be used without any change.

In the method according to the invention, methacrolein and methacrylic acid are produced through gas-phase contact oxidation of at least one raw material selected from the group consisting of isobutylene, TBA, and MTBE with molecular oxygen in the presence of a catalyst containing molybdenum, bismuth, and iron, using a fixed-bed reactor. In the production of methacrolein and methacrylic acid using the fixed-bed reactor, a reaction gas composed of the raw material, molecular oxygen, and the like is passed through the reaction tube packed with the catalyst. In the invention, the reaction is started at a temperature lower than the reaction temperature TA (° C.). However, since the activity of the catalyst is lowered with time, the reaction is controlled while increasing the reaction temperature such that the reaction rate of the raw material is kept constant, and the reaction is ended at a temperature exceeding TA (° C.).

As the fixed-bed reactor, a single tubular reactor equipped with one reaction tube may be used, but from the industrial point of view, a multitubular reactor equipped with a plurality of reaction tubes is preferably used. Further, as the reactor, a multitubular heat exchanger having an overall heat transfer coefficient of 5 to 250 W/(m²·K) is generally used.

From the viewpoint of easy management and easy purification process of a product to be obtained later, the reaction rate of the raw material is set to be constant throughout the entire reaction. On the occasion of setting the reaction rate to be constant, the reaction rate to be intended (hereinafter, referred to as a target reaction rate) can be appropriately determined according to properties of the catalyst (for example, selectivity to a target product with respect to the target reaction rate) or the like, but the target reaction rate is preferably from 90.0% to 99.9%, and more preferably from 92.0% to 99.5%. When the reaction rate is 90.0% or more, the yield of methacrolein or the like is improved. On the other hand, when the reaction rate is 99.9% or less, the reaction is easily started at TA or lower and it is possible to suppress decrease in the yield (selectivity) due to successive oxidation of methacrolein or the like.

Incidentally, in the invention, if the actual reaction rate is controlled to be in the range of ±1% with respect to the target reaction rate, it is assumed that the reaction rate is constant. Further, in a case where TBA and/or MTBE is used as the raw material, these raw materials are decomposed rapidly in isobutylene on a catalyst containing molybdenum, bismuth, and iron and thus it is considered that the reaction rate of the raw material is the reaction rate of isobutylene. The catalyst can be used while diluted with an inert substance such as silica, alumina, silica-alumina, magnesia, titania, or silicon carbide, as necessary. One kind of these may be used, or two or more kinds thereof may be used in combination.

In the method according to the invention, when the average of rates of temperature increase until the reaction temperature reaches TA is denoted by A (° C./hr) and the average of rates of temperature increase in the reaction temperature exceeding TA is denoted by B (° C./hr), the ratio (A/B) of A and B is 0.05 to 0.18. The A/B is preferably 0.06 to 0.17, more preferably 0.07 to 0.16, and still more preferably 0.08 to 0.15.

In the production of methacrolein or the like using a catalyst containing molybdenum, bismuth, and iron with a fixed-bed reactor in which the reaction is controlled while increasing the reaction temperature such that the reaction rate of the raw material is kept constant, there is a tendency that the yield (selectivity) of a target product is changed depending on the reaction temperature and the yield (selectivity) is improved as the reaction temperature is increased. Further, there is a tendency that the activation energy becomes larger in the region of a reaction temperature lower than TA (° C.), and the activation energy becomes smaller in the range of a reaction temperature higher than TA (° C.) with a boundary temperature TA (° C.) of the activation energy being a boundary. In other words, in order to maintain the reaction rate of the raw material, it is necessary to increase a degree of increasing the reaction temperature in the region of the reaction temperature exceeding the boundary temperature TA (° C.) of the activation energy rather than the region of the reaction temperature lower than the boundary temperature TA (° C.) of the activation energy.

From such a circumstance, in the range of the A/B less than 0.05, there is a case where the period of using the catalyst at a reaction temperature lower than the boundary temperature TA (° C.) of the activation energy becomes longer as a result, and thus the yield (selectivity) of methacrolein or the like is decreased, which is not favorable. On the other hand, in the range of the A/B exceeding 0.18, the period of using the catalyst at a high temperature exceeding the boundary temperature TA (° C.) of the activation energy becomes longer and thus this is advantageous to the yield (selectivity) of methacrolein or the like. However, as described above, the degree of increasing the reaction temperature for maintaining the reaction rate becomes larger in the region of the reaction temperature higher than TA (° C.) rather than the region of the reaction temperature lower than TA (° C.). Therefore, it is necessary to increase a reaction pressure or a molar ratio (O/R) of molecular oxygen and a raw material, which will be described later, in order to maintain the reaction rate to a predetermined value. When these values are excessively increased, the successive oxidation of methacrolein or the like proceeds and thus the yield (selectivity) is decreased, which is not favorable. Further, in a case where the reaction pressure or the like is not changed considerably, when the reaction rate of the raw material is intended to be maintained to a target value, the lifetime of the catalyst is decreased and thus it is difficult to use the catalyst for a planned period of time. Furthermore, when the target reaction rate is decreased in order to use the catalyst for a planned period of time, the yield of methacrolein or the like is decreased, which is not favorable.

In the method according to the invention, in order to adjust the A/B to be in the above range, for example, the adjustment can be performed by appropriately adjusting the catalyst activity, the reaction pressure, the molar ratio (O/R) of the molecular oxygen and the raw material, the contact time, or the like. The detailed description will be presented later. Incidentally, from the viewpoint that the reaction can be performed under constant conditions, it is preferable that the catalyst activity and the contact time be adjusted while the reaction pressure, and the molar ratio (O/R) of the molecular oxygen and the raw material are not changed from values set at the time of the reaction start such that the A/B is in the above range, and then methacrolein or the like be produced.

In the method according to the invention, A is preferably from 1.00×10⁻⁴ (° C./hr) to 9.00×10⁻⁴ (° C./hr), more preferably from 1.10×10⁻⁴ (° C./hr) to 8.90×10⁻⁴ (° C./hr), and still more preferably from 1.20×10⁻⁴ (° C./hr) to 8.80×10⁻³ (° C./hr). When A is too small, the catalyst is used for a long period of time at a low reaction temperature and thus there is a case where a disadvantage may be brought from the viewpoint of the yield of methacrolein. Further, when A is too large, the reaction temperature is increased but a disadvantage may be brought from the viewpoint of the lifetime of the catalyst. In addition to this, in a case where the reaction conditions are changed in order to maintain the operation period, there is a case where a disadvantage may also be brought from the viewpoint of the yield of methacrolein. Furthermore, from the same viewpoint, B is preferably from 5.00×10⁻⁴ (° C./hr) to 2.00×10⁻² (° C./hr), and more preferably from 1.00×10⁻³ (° C./hr) to 1.00×10″² (° C./hr).

Incidentally, A and B are values calculated by the following equations, respectively.

A (° C./hr)=(TA (° C.)−reaction start temperature (° C.))/(time (hr) from the start of the reaction until the reaction temperature reaches TA)

B (° C./hr)=(reaction end temperature (° C.)−TA (° C.))/(time (hr) from the instant at which the reaction temperature reaches TA to the end of the reaction)

Further, in the invention, since the activity of the catalyst is lowered with time, the reaction is controlled while increasing the reaction temperature such that the reaction rate of the raw material is kept constant. Therefore, the reaction temperature is not decreased during the reaction until the reaction conditions are extremely changed.

In the method according to the invention, when a time from the start of the reaction until the reaction temperature reaches TA (° C.) is denoted by C (hr) and a time from the instant at which the reaction temperature reaches TA (° C.) to the end of the reaction is denoted by D (hr), the ratio (C/D) of C and D is preferably 2.0 to 9.0. The ratio (C/D) is more preferably 2.3 to 8.0, and still more preferably 2.6 to 5.0. When the C/D is less than 2.0, the period of using the catalyst at a reaction temperature lower than the boundary temperature TA (° C.) of the activation energy becomes longer and thus the yield (selectivity) of methacrolein or the like may be decreased in some cases. On the other hand, when the C/D is more than 9.0, the period of using the catalyst at a high temperature exceeding the boundary temperature TA (° C.) of the activation energy becomes longer and thus this is advantageous to the yield (selectivity) of methacrolein or the like. However, as described above, the degree of increasing the reaction temperature for maintaining the reaction rate becomes larger in the region of the reaction temperature higher than TA (° C.) rather than the region of the reaction temperature lower than TA (° C.). Therefore, it is necessary to increase a reaction pressure or a molar ratio (O/R) of molecular oxygen and a raw material, which will be described later, in order to maintain the reaction rate to a predetermined value. When these values are excessively increased, the successive oxidation of methacrolein or the like proceeds and thus the yield (selectivity) may be decreased in some cases. Further, in a case where the reaction pressure or the like is not changed considerably, when the reaction rate of the raw material is intended to be maintained to a target value, the lifetime of the catalyst is decreased and thus it may be difficult to use the catalyst for a planned period of time in some cases. Furthermore, when the target reaction rate is decreased in order to use the catalyst for a planned period of time, the yield of methacrolein or the like may be decreased in some cases.

In the method according to the invention, from the viewpoint of the yield (selectivity) of methacrolein or the like and lifetime, C is preferably from 12800 (hr) to 39800 (hr), and more preferably from 13300 (hr) to 38400 (hr). Moreover, from the same viewpoint, D is preferably from 1590 (hr) to 11600 (hr), and more preferably from 2150 (hr) to 10600 (hr).

Further, the period of using the catalyst that is a sum (C+D) of C and D can be appropriately set depending on properties (such as activity and deterioration resistance) of the catalyst to be used, but is preferably from 17520 (hr) to 43800 (hr) and more preferably from 21900 (hr) to 39420 (hr). When C+D is 17520 (hr) or longer, the cost of the catalyst per unit mass of methacrolein or the like to be produced is decreased and thus this is economically advantageous. In addition, when C+D is 43800 (hr) or shorter, the catalyst can be used in a reaction temperature region where the yield (selectivity) of methacrolein or the like is favorable and thus this is economically advantageous.

In the method according to the invention, a temperature at which the reaction is started (reaction start temperature) is preferably from TA−15 (° C.) to TA−5 (° C.), more preferably from TA−13 (° C.) to TA−7 (° C.), and still more preferably from TA−12 (° C.) to TA−8 (° C.). When the reaction start temperature is set to be TA−15 (° C.) or higher, the period of using the catalyst at a low reaction temperature becomes shorter and thus the yield (selectivity) of methacrolein or the like is improved. Moreover, when the reaction start temperature is set to be TA−5 (° C.) or lower, it is not necessary to excessively increase the reaction pressure or the like in order to maintain the reaction rate of the raw material, and thus the yield (selectivity) is not decreased and the lifetime of the catalyst also becomes longer. In addition, it is also not necessary to decrease a target reaction rate.

In the method according to the invention, the reaction end temperature can be set depending on usage conditions of the catalyst, or the like. For example, the reaction end temperature can be set to be a temperature at which the increase rate becomes larger such that the reaction rate of the raw material cannot be kept constant. Further, in the method according to the invention, as described above, there is a tendency that the yield (selectivity) is improved as the reaction temperature is increased. However, although the improvement of the yield (selectivity) varies depending on properties of the catalyst to be used, there is a tendency that, in a case where the reaction temperature is in the range until the reaction temperature reaches TA+10 (° C.) and is from TA+10 (° C.) to TA+30 (° C.), the yield is almost the same and, in a case where the reaction temperature is TA+50 (° C.) or higher, the yield is decreased. Therefore, from the viewpoint of improving or maintaining the yield while the operation period is extended, the reaction end temperature is preferably from TA+10 (° C.) to TA+50 (° C.), and more preferably from TA+10 (° C.) to TA+30 (° C.). Furthermore, as a heat medium provided in the fixed-bed reactor and used for controlling the reaction temperature, niter is generally used. Since the decomposition temperature of the niter is about 400° C., the reaction end temperature is preferably 400° C. or lower.

As described above, in the method according to the invention, as the method by which the A/B is set to be within the above range, for example, a method of appropriately adjusting catalyst activity, a reaction pressure, a molar ratio (0/R) of molecular oxygen and a raw material, a contact time, and the like is exemplified. The adjustment thereof can be appropriately achieved by those skilled in the art.

In the adjustment of the catalyst activity, for example, according to JP 2004-13021 A, there are (1) a method of changing calcination conditions, (2) a method of changing a kind and/or an amount of an alkali metal, and (3) a method of changing a mixing time of a solution or dispersion containing catalyst components, a stirring condition at the time of heating or aging, and the like in a preparation process of a mixed slurry of catalyst components. In the method according to the invention, it is preferable that the adjustment of the catalyst activity be carried out by (1) the method of changing calcination conditions. Specifically, for example, a calcination temperature, a calcination time, presence and absence of primary calcination, a time point of calcination before or after molding, or the like is changed. Further, when the adjustment of the catalyst activity is performed by (2) the method of changing a kind and/or an amount of an alkali metal, there is a case where the behavior of lowering in the catalyst activity with time is changed depending on a kind and/or an amount of an alkali metal. Furthermore, when the adjustment of the catalyst activity is performed by (3) the method of changing a mixing time of a solution or dispersion containing catalyst components, a stirring condition at the time of heating or aging, and the like in a preparation process of a mixed slurry of catalyst components, there is a case where the reproducibility of the adjustment of the catalyst activity is decreased.

The reaction pressure is preferably from 20 kPaG to 200 kPaG (gauge pressure: Hereinafter, all of pressures are expressed in the gauge pressure) as an average pressure of inlet pressure and outlet pressure of the reaction tube, and more preferably from 50 kPaG to 150 kPaG. When the reaction pressure is set to be 20 kPaG or more, the lifetime of the catalyst does not deteriorate even if the reaction rate of the raw material is intended to be kept to a target reaction rate. Moreover, when the reaction pressure is set to be 200 kPaG or less, the successive oxidation of methacrolein or the like is suppressed and thus the yield (selectivity) is improved.

The molar ratio (O/R) of molecular oxygen and a raw material is preferably from 0.5 to 3.0, and more preferably from 1.0 to 2.5. When the O/R is set to be 0.5 or more, the lifetime of the catalyst does not deteriorate even if the reaction rate of the raw material is intended to be kept to a target reaction rate. Moreover, when the O/R is set to be 3.0 or less, the successive oxidation of methacrolein or the like is suppressed and thus the yield (selectivity) is improved. In addition, it is possible to avoid the reaction from falling within an explosion range.

The contact time is preferably from 1.5 seconds to 15 seconds, and more preferably from 2.5 seconds to 10 seconds.

In the method according to the invention, the concentration of a raw material in the reaction gas can be appropriately determined from the range of a well-known condition, but is preferably 1 to 10% by volume. Further, as the source of molecular oxygen, it is economical to use air but air enriched with pure oxygen can also be used if necessary. It is preferable that inert gas such as nitrogen for dilution be included in the reaction gas. Moreover, the reaction gas may also contain water vapor.

### EXAMPLE

Hereinafter, examples of the invention will be described, but the invention is not limited to these examples. In this specification, “part” represents part by mass. The analyses of reaction products were carried out with gas chromatography. Further, a reaction rate of isobutylene as a raw material and selectivities to methacrolein and methacrylic acid to be produced are defined as follows. Incidentally, E represents the number of moles of the raw material reacted, F represents the number of moles of the raw material supplied, G represents the number of moles of methacrolein produced, and H represents the number of moles of methacrylic acid produced.

Reaction rate of raw material (%)=E/F×100

Selectivity to methacrolein (%)=G/E×100

Selectivity to methacrylic acid (%)=H/E×100

Further, the average reaction rate, the average selectivity to methacrolein, and the average selectivity to methacrylic acid are values obtained by measuring the reaction rates of the raw material, the selectivities to methacrolein, and the selectivities to methacrylic acid per 24 hr and calculating each average thereof from the start of the reaction to the end of the reaction.

### Example 1

### Production of Catalyst

To 6000 parts of pure water, 3000 parts of ammonium paramolybdate was dissolved. Subsequently, 74.4 parts of ammonium paratungstate, 138.0 parts of cesium nitrate, 164.4 parts of antimony trioxide, and 198.0 parts of bismuth trioxide were added to the solution while stirring, and the resultant mixture was heated to 50° C. (liquid A). Separately, to 6000 parts of pure water, 1258.8 parts of ferric nitrate, 453.0 parts of nickel nitrate, 2719.8 parts of cobalt nitrate, 187.8 parts of lead nitrate, and 33.6 parts of 85% phosphoric acid were sequentially added and dissolved, and the resultant mixture was heated to 30° C. (liquid B). The liquid B was added to the liquid A under stirring to obtain an aqueous slurry. The aqueous slurry was aged at 90° C. for 2 hours. Thereafter, the aqueous slurry was heated to 103° C. and concentrated for 1 hour, and then dried using a spray dryer to obtain dry powder. The obtained dry powder was primarily calcined at 300° C. for 1 hour and then secondarily calcined at 510° C. for 3 hours to obtain calcined catalyst powder.

After 4000 parts of the obtained calcined catalyst powder and 120 parts of methylcellulose powder were mixed well with each other, 1440 parts of pure water was added thereto and the resultant mixture was kneaded by a kneading machine until the mixture became a clayey substance. Subsequently, the obtained amorphous kneaded product was subjected to extrusion molding by using a screw-type extrusion molding machine to obtain a columnar primary molded article having a diameter of 45 mm and a length of 280 mm. This primary molded article was molded by using a piston-type extrusion molding machine to be formed in a ring shape with an outer diameter of 5 mm, an inner diameter of 2 mm, and a length of 5 mm. The obtained molded article was dried at 110° C. using a hot-air dryer and was calcined again at 400° C. for 3 hours to obtain a catalyst.

The composition of elements other than oxygen in the obtained catalyst was Mo₁₂Bi_{0.6}Fe_{2.2}Co_{6.6}Ni_{1.1}Pb_{0.4}P_{0.2}W_{0.2}Sb_{0.8}Cs_{0.5}. Incidentally, the composition of the catalyst is a value calculated from an amount of the charged raw material of each element.

(Determination of Boundary Temperature TA of Activation Energy of Reaction)

To a stainless steel reaction tube which has an inner diameter of 27.5 mm and a height of 4 m and is equipped with a thermo-bath outside, 2000 g of the obtained catalyst was packed. Subsequently, a reaction gas composed of 5% by volume of isobutylene, 12% by volume of oxygen, 10% by volume of water vapor, and 73% by volume of nitrogen was passed through the catalyst layer at a reaction pressure of 100 kPaG and a contact time of 3.5 seconds. The reaction temperature (temperature at which a heat medium is introduced to the thermo-bath) was changed in the range of 315 to 375° C. at an interval of 2 to 5° C., the gas-phase contact oxidation of isobutylene was performed, and the activation energy was calculated from the reaction rate of isobutylene at each temperature. As a result, the boundary temperature TA (° C.) of the activation energy was 330° C., the activation energy of the temperature range lower than TA was 105 kJ/mol, and the activation energy of the temperature range higher than TA was 36 kJ/mol.

(Production of Methacrolein and Methacrylic Acid)

To the reaction tube used in the above-described determination of TA, 2000 g of the obtained catalyst was packed. Subsequently, a reaction gas composed of 5% by volume of isobutylene (reaction raw material), 11% by volume of oxygen, 10% by volume of water vapor, and 74% by volume of nitrogen was passed through the catalyst layer at a reaction pressure of 100 kPaG and a contact time of 4.5 seconds. The reaction start temperature (thermo-bath initial temperature) of gas-phase contact oxidation of isobutylene was set to be 318° C. The target reaction rate of the raw material was set to be 95%, and when the reaction rate was decreased by lowering in catalyst activity with time, the reaction rate was kept almost constant by increasing the reaction temperature (temperature at which a heat medium is introduced to the thermo-bath). According to this method, the gas-phase contact oxidation of isobutylene was performed until the reaction temperature reached 360° C.

The time C (hr) from the start of the reaction until the reaction temperature reached TA was 16900 hr, the time D (hr) from the instant at which the reaction temperature reached TA to the end of the reaction was 5600 hr, and the C/D was 3.0. Further, the ratio AB of the average A (° C./hr) of rates of temperature increase until the reaction temperature reached TA and the average B (° C./hr) of rates of temperature increase in the reaction temperature exceeding TA was 0.13. The average reaction rate of isobutylene in the period of using the catalyst was 95.1%, the average selectivity to methacrolein was 87.8%, the average selectivity to methacrylic acid was 5.5%, and the average of the total yield of methacrolein and methacrylic acid was 88.7%. The results are presented in Table 1.

### Comparative Example 1

### Production of Catalyst

The catalyst was produced by the same method as in Example 1, except that the secondary calcination temperature was changed to 512° C.

(Determination of Boundary Temperature TA of Activation Energy of Reaction)

When TA was measured by the same method as in Example 1, TA (° C.) was 330° C.

(Production of Methacrolein and Methacrylic Acid)

To the reaction tube used in Example 1, 2150 g of the obtained catalyst was packed. Subsequently, a reaction gas composed of 5% by volume of isobutylene (reaction raw material), 11% by volume of oxygen, 10% by volume of water vapor, and 74% by volume of nitrogen was passed through the catalyst layer at a reaction pressure of 100 kPaG and a contact time of 4.8 seconds. The target reaction rate of the raw material was set to be 95%, and the gas-phase contact oxidation of isobutylene was performed by the same method as in Example 1.

The reaction start temperature of the gas-phase contact oxidation of isobutylene was set to be 318° C. However, the time C (hr) until the reaction temperature reached TA was 20300 hr and was longer than that in Example 1. Here, the reaction was carried out until the reaction temperature reached 360° C. while, in the region of the reaction temperature exceeding TA, the oxygen concentration was changed to 10% by volume, the nitrogen concentration was changed to 75% by volume, and the reaction pressure was changed to 85 kPaG with no change in the target reaction rate of 95%. The time D (hr) from the instant at which the reaction temperature reached TA to the end of the reaction was 2200 hr and the C/D was 9.2. Incidentally, the period of the entire reaction was 22500 hr as long as that in Example 1.

In addition, the ratio A/B of the average A (° C./hr) of rates of temperature increase until the reaction temperature reached TA and the average B (° C./hr) of rates of temperature increase in the reaction temperature exceeding TA was 0.04. The average reaction rate of isobutylene in the period of using the catalyst was 95.4%, the average selectivity to methacrolein was 86.0%, the average selectivity to methacrylic acid was 4.8%, and the average of the total yield of methacrolein and methacrylic acid was 86.6%. The results are presented in Table 1. As the period of using the catalyst at a low reaction temperature became long, the yields of methacrolein and methacrylic acid decreased.

### Comparative Example 2

### Production of Catalyst

The catalyst was produced by the same method as in Example 1, except that the secondary calcination temperature was changed to 508° C.

(Determination of Boundary Temperature TA of Activation Energy of Reaction)

When TA was measured by the same method as in Example 1, TA (° C.) was 330° C.

(Production of Methacrolein and Methacrylic Acid)

To the reaction tube used in Example 1, 1850 g of the obtained catalyst was packed. Subsequently, a reaction gas composed of 5% by volume of isobutylene (reaction raw material), 11% by volume of oxygen, 10% by volume of water vapor, and 74% by volume of nitrogen was passed through the catalyst layer at a reaction pressure of 100 kPaG and a contact time of 4.2 seconds. The target reaction rate of the raw material was set to be 95%, and the gas-phase contact oxidation of isobutylene was performed by the same method as in Example 1.

The reaction start temperature of the gas-phase contact oxidation of isobutylene was set to be 318° C. However, the time C (hr) until the reaction temperature reached TA was 13000 hr and was shorter than that in Example 1. Here, the reaction was carried out until the reaction temperature reached 360° C. while, in the region of the reaction temperature exceeding TA, the oxygen concentration was changed to 12% by volume, the nitrogen concentration was changed to 73% by volume, and the reaction pressure was changed to 115 kPaG with no change in the target reaction rate of 95%. The time D (hr) from the instant at which the reaction temperature reached TA to the end of the reaction was 7000 hr and the C/D was 1.9. Incidentally, the period of the entire reaction was 20000 hr, that is, the period became shorter than that in Example 1.

In addition, the ratio A/B of the average A (° C./hr) of rates of temperature increase until the reaction temperature reached TA and the average B (° C./hr) of rates of temperature increase in the reaction temperature exceeding TA was 0.22. The average reaction rate of isobutylene in the period of using the catalyst was 95.2%, the average selectivity to methacrolein was 86.8%, the average selectivity to methacrylic acid was 5.1%, and the average of the total yield of methacrolein and methacrylic acid was 87.5%. The results are presented in Table 1. Although the rate A of temperature increase until the reaction temperature reached TA was large, and the molar ratio of molecular oxygen and isobutylene and the reaction pressure in the region of the reaction temperature exceeding TA were increased, the catalyst could not be used in the same period as that in Example 1. Further, the yields of methacrolein and methacrylic acid also decreased.

### Comparative Example 3

### Production of Catalyst

The catalyst was produced by the same method as in Example 1, except that the secondary calcination temperature was changed to 508° C.

(Determination of Boundary Temperature TA of Activation Energy of Reaction)

When TA was measured by the same method as in Example 1, TA (° C.) was 330° C.

(Production of Methacrolein and Methacrylic Acid)

To the reaction tube used in Example 1, 1850 g of the obtained catalyst was packed. Subsequently, a reaction gas composed of 5% by volume of isobutylene (reaction raw material), 11% by volume of oxygen, 10% by volume of water vapor, and 74% by volume of nitrogen was passed through the catalyst layer at a reaction pressure of 100 kPaG and a contact time of 4.2 seconds. The target reaction rate of the raw material was set to be 95%, and the gas-phase contact oxidation of isobutylene was performed by the same method as in Example 1.

The reaction start temperature of the gas-phase contact oxidation of isobutylene was set to be 318° C. However, the time C (hr) until the reaction temperature reached TA was 13000 hr and was shorter than that in Example 1. Here, the reaction was carried out until the reaction temperature reached 360° C. while, in the region of the reaction temperature exceeding TA, the oxygen concentration was changed to 12% by volume, the nitrogen concentration was changed to 73% by volume, and the reaction pressure was changed to 115 kPaG, and the target reaction rate was changed to 90.0% so as not to shorten the period of the entire reaction. As a result, the time D (hr) from the instant at which the reaction temperature reached TA to the end of the reaction was 9500 hr and the C/D was 1.4. Incidentally, the period of the entire reaction was 22500 hr as long as that in Example 1.

In addition, the ratio A/B of the average A (° C./hr) of rates of temperature increase until the reaction temperature reached TA and the average B (° C./hr) of rates of temperature increase in the reaction temperature exceeding TA was 0.29. The average reaction rate of isobutylene in the period of using the catalyst was 93.1%, the average selectivity to methacrolein was 87.5%, the average selectivity to methacrylic acid was 5.2%, and the average of the total yield of methacrolein and methacrylic acid was 86.3%. The results are presented in Table 1. Since the rate A of temperature increase until the reaction temperature reached TA was large, and the catalyst could not be used in the same period as that in Example 1 without increasing the molar ratio of molecular oxygen and isobutylene and the reaction pressure in the region of the reaction temperature exceeding TA and decreasing the target reaction rate, the yields of methacrolein and methacrylic acid decreased.

### Example 2

### Production of Methacrolein and Methacrylic Acid

To the reaction tube used in Example 1, 2150 g of the catalyst produced in Comparative Example 1 was packed. Subsequently, a reaction gas composed of 5% by volume of isobutylene (reaction raw material), 10.5% by volume of oxygen, 10% by volume of water vapor, and 74.5% by volume of nitrogen was passed through the catalyst layer at a reaction pressure of 95 kPaG and a contact time of 4.8 seconds. The target reaction rate of the raw material was set to be 95%, and the gas-phase contact oxidation of isobutylene was performed by the same method as in Example 1. Incidentally, the reaction start temperature of the gas-phase contact oxidation of isobutylene was set to be 320° C.

The time C (hr) from the start of the reaction until the reaction temperature reached TA was 18500 hr, the time D (hr) from the instant at which the reaction temperature reached TA to the end of the reaction was 4000 hr, and the C/D was 4.6. Incidentally, the period of the entire reaction was 22500 hr as long as that in Example 1. Further, the ratio A/B of the average A (° C./hr) of rates of temperature increase until the reaction temperature reached TA and the average B (° C./hr) of rates of temperature increase in the reaction temperature exceeding TA was 0.07. The average reaction rate of isobutylene in the period of using the catalyst was 95.0%, the average selectivity to methacrolein was 87.5%, the average selectivity to methacrylic acid was 5.2%, and the average of the total yield of methacrolein and methacrylic acid was 88.1%. The results are presented in Table 1.

### Example 3

### Production of Methacrolein and Methacrylic Acid

To the reaction tube used in Example 1, 2150 g of the catalyst produced in Comparative Example 1 was packed. Subsequently, a reaction gas composed of 5% by volume of isobutylene (reaction raw material), 10% by volume of oxygen, 10% by volume of water vapor, and 75% by volume of nitrogen was passed through the catalyst layer at a reaction pressure of 90 kPaG and a contact time of 4.8 seconds. The target reaction rate of the raw material was set to be 95%, and the gas-phase contact oxidation of isobutylene was performed by the same method as in Example 1.

The reaction start temperature of the gas-phase contact oxidation of isobutylene was set to be 321° C. The time C (hr) until the reaction temperature reached TA was 16500 hr, the time D (hr) from the instant at which the reaction temperature reached TA to the end of the reaction was 6000 hr, and the C/D was 2.8. Incidentally, the period of the entire reaction was 22500 hr as long as that in Example 1.

In addition, the ratio A/B of the average A (° C./hr) of rates of temperature increase until the reaction temperature reached TA and the average B (° C./hr) of rates of temperature increase in the reaction temperature exceeding TA was 0.11. The average reaction rate of isobutylene in the period of using the catalyst was 95.3%, the average selectivity to methacrolein was 87.6%, the average selectivity to methacrylic acid was 5.4%, and the average of the total yield of methacrolein and methacrylic acid was 88.6%. The results are presented in Table 1.

### Example 4

### Production of Methacrolein and Methacrylic Acid

To the reaction tube used in Example 1, 1850 g of the catalyst produced in Comparative Example 2 was packed. Subsequently, a reaction gas composed of 5% by volume of isobutylene (reaction raw material), 12% by volume of oxygen, 10% by volume of water vapor, and 73% by volume of nitrogen was passed through the catalyst layer at a reaction pressure of 110 kPaG and a contact time of 4.2 seconds. The target reaction rate of the raw material was set to be 95%, and the gas-phase contact oxidation of isobutylene was performed by the same method as in Example 1.

The reaction start temperature of the gas-phase contact oxidation of isobutylene was set to be 315° C. The time C (hr) until the reaction temperature reached TA was 17100 hr, the time D (hr) from the instant at which the reaction temperature reached TA to the end of the reaction was 5400 hr, and the C/D was 3.2. Incidentally, the period of the entire reaction was 22500 hr as long as that in Example 1.

In addition, the ratio A/B of the average A (° C./hr) of rates of temperature increase until the reaction temperature reached TA and the average B (° C./hr) of rates of temperature increase in the reaction temperature exceeding TA was 0.16. The average reaction rate of isobutylene in the period of using the catalyst was 95.0%, the average selectivity to methacrolein was 87.5%, the average selectivity to methacrylic acid was 5.2%, and the average of the total yield of methacrolein and methacrylic acid was 88.1%. The results are presented in Table 1.

### Example 5

### Production of Methacrolein and Methacrylic Acid

To the reaction tube used in Example 1, 2000 g of the catalyst produced in Example 1 was packed. Subsequently, the gas-phase contact oxidation was performed by the same method as in Example 1, except that the raw material was changed to TBA. As for the target reaction rate of the raw material, the target reaction rate of isobutylene was set to be 95% on the assumption that TBA is completely decomposed in isobutylene.

The reaction start temperature of the gas-phase contact oxidation was set to be 318° C. The time C (hr) until the reaction temperature reached TA was 16600 hr, the time D (hr) from the instant at which the reaction temperature reached TA to the end of the reaction was 5900 hr, and the C/D was 2.8. Incidentally, the period of the entire reaction was 22500 hr as long as that in Example 1.

In addition, the ratio A/B of the average A (° C./hr) of rates of temperature increase until the reaction temperature reached TA and the average B (° C./hr) of rates of temperature increase in the reaction temperature exceeding TA was 0.14. The average reaction rate of TBA in the period of using the catalyst was 100% (the average reaction rate of isobutylene was 95.0%), the average selectivity to methacrolein was 88.0%, the average selectivity to methacrylic acid was 5.6%, and the average of the total yield of methacrolein and methacrylic acid was 88.9%. The results are presented in Table 1. Incidentally, the average reaction rate in Table 1 represents the average reaction rate of isobutylene in a case where it is assumed that TBA is completely decomposed in isobutylene.

### Example 6

### Production of Methacrolein and Methacrylic Acid

To the reaction tube used in Example 1, 2000 g of the catalyst produced in Example 1 was packed. Subsequently, the gas-phase contact oxidation was performed by the same method as in Example 1, except that the raw material was changed to TBA and the pressure of the reaction gas was changed to 105 kPaG. As for the target reaction rate of the raw material, the target reaction rate of isobutylene was set to be 95% on the assumption that TBA is completely decomposed in isobutylene.

The reaction start temperature of the gas-phase contact oxidation was set to be 317° C. The time C (hr) until the reaction temperature reached TA was 18100 hr. Subsequently, the reaction was carried out until the reaction temperature reached 360° C. with no change in the reaction pressure while, in the region of the reaction temperature exceeding TA, the oxygen concentration was changed to 10.5% by volume and the nitrogen concentration was changed to 74.5% by volume with no change in the target reaction rate of 95%. The time D (hr) from the instant at which the reaction temperature reached TA to the end of the reaction was 4400 hr and the C/D was 4.1. Incidentally, the period of the entire reaction was 22500 hr as long as that in Example 1.

In addition, the ratio A/B of the average A (° C./hr) of rates of temperature increase until the reaction temperature reached TA and the average B (° C./hr) of rates of temperature increase in the reaction temperature exceeding TA was 0.11. The average reaction rate of TBA in the period of using the catalyst was 100% (the average reaction rate of isobutylene was 95.2%), the average selectivity to methacrolein was 87.4%, the average selectivity to methacrylic acid was 5.5%, and the average of the total yield of methacrolein and methacrylic acid was 88.4%. The results are presented in Table 1. Incidentally, the average reaction rate in Table 1 represents the average reaction rate of isobutylene in a case where it is assumed that TBA is completely decomposed in isobutylene.

TABLE 1

| | Catalyst | | | | Reaction | | | |
|---|---|---|---|---|---|---|---|---|
| | calcination | Reaction | Oxygen/raw | Contact | start | | | |
| | temperature | pressure | material | time | temperature | C | D | C + D |
| | (° C.) | (kPa) | (molar ratio) | (s) | (° C.) | (h) | (h) | (h) |
| Example 1 | 510 | 100 | 2.2 | 4.5 | 318 | 16900 | 5600 | 22500 |
| Comparative | 512 | 100 → 85 | 2.2 → 2.0 | 4.8 | 318 | 20300 | 2200 | 22500 |
| Example 1 | | | | | | | | |
| Comparative | 508 | 100 → 115 | 2.2 → 2.4 | 4.2 | 318 | 13000 | 7000 | 20000 |
| Example 2 | | | | | | | | |
| Comparative | 508 | 100 → 115 | 2.2 → 2.4 | 4.2 | 318 | 13000 | 9500 | 22500 |
| Example 3 | | | | | | | | |
| Example 2 | 512 | 95 | 2.1 | 4.8 | 320 | 18500 | 4000 | 22500 |
| Example 3 | 512 | 90 | 2.0 | 4.8 | 321 | 16500 | 6000 | 22500 |
| Example 4 | 508 | 110 | 2.4 | 4.2 | 315 | 17100 | 5400 | 22500 |
| Example 5 | 510 | 100 | 2.2 | 4.5 | 318 | 16600 | 5900 | 22500 |
| Example 6 | 510 | 105 | 2.2→2.1 | 4.5 | 317 | 18100 | 4400 | 22500 |

| | | | | | | | Average | |
|---|---|---|---|---|---|---|---|---|
| | | | | | Average | Average | selectivity to | |
| | | | | | reaction | selectivity to | methacrylic | Average |
| | | A | B | | rate | methacrolein | acid | yield |
| | C/D | (° C./h) | (° C./h) | A/B | (%) | (%) | (%) | (%) |
| Example 1 | 3.0 | 7.10 × 10⁻⁴ | 5.36 × 10⁻³ | 0.13 | 95.1 | 87.8 | 5.5 | 88.7 |
| Comparative | 9.2 | 5.91 × 10⁻⁴ | 1.36 × 10⁻² | 0.04 | 95.4 | 86.0 | 4.8 | 86.6 |
| Example 1 | | | | | | | | |
| Comparative | 1.9 | 9.23 × 10⁻⁴ | 4.29 × 10⁻³ | 0.22 | 95.2 | 86.8 | 5.1 | 87.5 |
| Example 2 | | | | | | | | |
| Comparative | 1.4 | 9.23 × 10⁻⁴ | 3.16 × 10⁻³ | 0.29 | 93.1 | 87.5 | 5.2 | 86.3 |
| Example 3 | | | | | | | | |
| Example 2 | 4.6 | 5.41 × 10⁻⁴ | 7.50 × 10⁻³ | 0.07 | 95.0 | 87.5 | 5.2 | 88.1 |
| Example 3 | 2.8 | 5.45 × 10⁻⁴ | 5.00 × 10⁻³ | 0.11 | 95.3 | 87.6 | 5.4 | 88.6 |
| Example 4 | 3.2 | 8.77 × 10⁻⁴ | 5.56 × 10⁻³ | 0.16 | 95.0 | 87.5 | 5.2 | 88.1 |
| Example 5 | 2.8 | 7.23 × 10⁻⁴ | 5.08 × 10⁻³ | 0.14 | 95.0 | 88.0 | 5.6 | 88.9 |
| Example 6 | 4.1 | 7.18 × 10⁻⁴ | 6.82 × 10⁻³ | 0.11 | 95.2 | 87.4 | 5.5 | 88.4 |

This application claims priority based on the Japanese Patent Application No. 2012-245263 filed on Nov. 7, 2012 and the disclosure of which is hereby incorporated in its entirety.

Hereinbefore, the invention has been described with reference to embodiments and examples. However, the invention is not limited to the above embodiments and examples. Various changes understandable to those skilled in the art can be made to the configuration and the specifics of the invention without departing from the scope of the invention.

1. A method for producing methacrolein and methacrylic acid through gas-phase contact oxidation of at least one raw material selected from the group consisting of isobutylene, tert-butyl alcohol, and methyl-tert-butyl ether with molecular oxygen in the presence of a catalyst containing molybdenum, bismuth, and iron, using a fixed-bed reactor,
wherein, when a boundary temperature of activation energy of the oxidation reaction is denoted by TA (° C.), the reaction is started at a temperature lower than the reaction temperature TA (° C.), the reaction is controlled while increasing a reaction temperature such that a reaction rate of the raw material is kept constant, and the reaction is ended at a reaction temperature exceeding TA (° C.), and
wherein, when an average of rates of temperature increase until the reaction temperature reaches TA (° C.) is denoted by A (° C./hr) and an average of rates of temperature increase in the reaction temperature exceeding TA (° C.) is denoted by B (° C./hr), a ratio (A/B) of A and B is 0.05 to 0.18.

2. The method for producing methacrolein and methacrylic acid according to claim 1, wherein the ratio (A/B) of A and B is 0.08 to 0.15.

3. The method for producing methacrolein and methacrylic acid according to claim 1, wherein a value of A is from 1.00×10⁻⁴ (° C./hr) to 9.00×10⁻⁴ (° C./hr).

4. The method for producing methacrolein and methacrylic acid according to claim 1, wherein, when a time from the start of the reaction until the reaction temperature reaches TA (° C.) is denoted by C (hr) and a time from the instant at which the reaction temperature reaches TA to the end of the reaction is denoted by D (hr), a ratio (C/D) of C and D is 2.0 to 9.0.

5. The method for producing methacrolein and methacrylic acid according to claim 4, wherein the ratio (C/D) of C and D is 2.6 to 5.0.

6. The method for producing methacrolein and methacrylic acid according to claim 1, wherein a temperature at which the reaction is started is from TA−15 (° C.) to TA−5 (° C.).

7. The method for producing methacrolein and methacrylic acid according to claim 1, wherein a temperature at which the reaction is ended is from TA+10 (° C.) to TA+50 (° C.).

8. The method for producing methacrolein and methacrylic acid according to claim 1, wherein the catalyst has the composition represented by the following Formula (1).
MoₐBi_{b}Fe_{c}M_{d}XₑY_{f}Z_{g}SiₕOᵢ (1)
(In Formula (1), Mo represents molybdenum, Bi represents bismuth, Fe represents iron, Si represents silicon, and O represents oxygen. M represents at least one element selected from the group consisting of cobalt and nickel. X represents at least one element selected from the group consisting of chromium, lead, manganese, calcium, magnesium, niobium, silver, barium, tin, tantalum, and zinc. Y represents at least one element selected from the group consisting of phosphorus, boron, sulfur, selenium, tellurium, cerium, tungsten, antimony, and titanium. Z represents at least one element selected from the group consisting of lithium, sodium, potassium, rubidium, cesium, and thallium. Each of a, b, c, d, e, f, g, h, and i represents an atomic ratio of each element, and when a is 12, b is 0.01 to 3, c is 0.01 to 5, d is 1 to 12, e is 0 to 8, f is 0 to 5, g is 0.001 to 2, and h is 0 to 20. i is an atomic ratio of oxygen necessary for satisfying a valence of each of the components.)
